(19) United States
(12) Patent Application Publication
(10) Pub. No.: US 2024/0415471 A1
(43) Pub. Date: Dec. 19, 2024

(54) BIOPSY EQUIPMENT, SUPPORT METHOD AND BIOPSY DEVICE

(71) Applicant: Siemens Healthineers AG, Forchheim (DE)

(72) Inventor: Christian HAESSLER, Bubenreuth (DE)

(73) Assignee: Siemens Healthineers AG, Forchheim (DE)

(21) Appl. No.: 18/742,093

(22) Filed: Jun. 13, 2024

(30) Foreign Application Priority Data
Jun. 15, 2023 (EP) 23179521.2

(51) Int. Cl. A61B 6/02; A61B 6/04; A61B 6/12; A61B 10/04

(52) U.S. Cl. CPC A61B 6/025; A61B 6/0414; A61B 6/0435; A61B 6/0492; A61B 6/12; A61B 2010/045

(57) ABSTRACT

One or more example embodiments relates to biopsy equipment for performing a biopsy on a female breast, comprising imaging equipment for tomosynthesis of the breast with compression equipment configured to fix the breast, a hand-held biopsy device with a biopsy needle, wherein the biopsy device comprises a position sensor for measuring position information indicating the orientation of the biopsy needle, marking equipment registered with the imaging equipment for marking an entry point for the biopsy needle on the breast fixed in the compression equipment according to planning information for a biopsy to be performed based on image data from the imaging equipment, outputting equipment for outputting guidance information indicating the relative orientation of the biopsy needle according to the position information to a target orientation according to the planning information for a person performing the biopsy, and control equipment for controlling the biopsy equipment according to the planning information.

### CROSS-REFERENCE TO RELATED APPLICATION(S)

The present application claims priority under 35 U.S.C. § 119 to European Patent Application No. 23179521.2, filed Jun. 15, 2023, the entire contents of which is incorporated herein by reference.

### FIELD

One or more example embodiments relates to biopsy equipment for performing a biopsy on a female breast comprising imaging equipment for tomosynthesis of the breast with compression equipment for fixing the breast. In addition, one or more example embodiments relates to a method for supporting a person performing a biopsy with biopsy equipment and a biopsy device.

### RELATED ART

Biopsies are usually performed on the female breast (mammary gland) or other parts of the human body in order to be able to examine anatomical abnormalities, in particular tissue changes, in more detail and produce a subsequent diagnosis. This is due to the fact that medical imaging, for example breast tomosynthesis, is often not sufficient to enable a conclusive diagnosis. In a biopsy, a biopsy needle is used as a medical instrument in order to access the region of interest as minimally invasively as possible and take a tissue sample there. For example, the so-called punch biopsy method is known for this purpose.

In biopsies, it is essential to hit the region of interest within the patient as exactly as possible; this requires the biopsy needle to be positioned in three-dimensional space with sufficient positioning accuracy. For this purpose, biopsy equipment usually comprises imaging equipment, usually X-ray equipment. In many cases, the imaging equipment is tomosynthesis equipment so that screening can also be performed on the biopsy equipment. Herein, numerous requirements are placed on biopsy equipment. With regard to imaging, image artifacts from the biopsy needle and components of a biopsy unit containing the biopsy needle should be avoided as far as possible during X-ray recording, both in two-dimensional X-ray recording and three-dimensional X-ray recording, in particular tomosynthesis recording. The biopsy device and other components to be positioned close to the patient should occupy as little space as possible on and around the object-support table in order not to restrict access to the patient and the examination region. The duration of treatment should be as short as possible. For example, this requires that motorized positioning movements should be of short duration. Finally, low weight and low costs are desirable.

The prior art has already proposed embodiments of biopsy equipment in which a biopsy unit comprising actuators for positioning and moving the biopsy needle is attached to the object-support table and/or the stand of the imaging equipment. Herein, the entire biopsy unit is registered with the coordinate system of the imaging equipment, so that planning information obtained with the aid of image data from the imaging equipment can be converted directly into an actuation of the actuators, which are in particular embodied as electric motors, in order to perform tissue removal completely automatically.

### SUMMARY

However, such an embodiment is disadvantageous in that such permanently installed or permanently installable biopsy units are expensive, occupy a large amount of installation space and have to be implemented in a reliable and robust highly complex manner such that the region of interest is hit with sufficient accuracy. Moreover, patient acceptance is sometimes limited since the biopsy is performed fully automatically without the involvement of a person who inspires trust.

Therefore, one or more example embodiments provides biopsy equipment that allows a highly accurate biopsy in a simple manner despite being implemented in a cost-effective and space-saving manner.

This is achieved by the provision of biopsy equipment, a method and a biopsy device according to the independent claims. Advantageous embodiments result from the subclaims.

### BRIEF DESCRIPTION OF THE DRAWINGS

Further advantages and details emerge from the exemplary embodiments described below and from the drawings. The drawings show:

FIG. 1 illustrates functional components of biopsy equipment according to one or more example embodiments,

FIG. 2 is a perspective view of a biopsy device according to one or more example embodiments,

FIG. 3 is a view of part of imaging equipment according to one or more example embodiments,

FIG. 4 illustrates a compression plate according to one or more example embodiments,

FIG. 5 is a graph to explain the actuation of output means according to one or more example embodiments, and

FIG. 6 illustrates a visual representation on a display according to one or more example embodiments.

### DETAILED DESCRIPTION

According to one or more example embodiments, biopsy equipment for performing a biopsy on a female breast comprises:
imaging equipment for tomosynthesis of the breast with compression equipment for fixing the breast,
a hand-held biopsy device with a biopsy needle, wherein the biopsy device comprises a position sensor for measuring position information indicating the orientation of the biopsy needle,
marking equipment registered with the imaging equipment for marking an entry point for the biopsy needle on the breast fixed in the compression equipment according to planning information for a biopsy to be performed based on image data from the imaging equipment,
outputting equipment for outputting guidance information indicating the relative orientation of the needle according to the position information to a target orientation according to the planning information for a person performing the biopsy, and
control equipment for controlling the biopsy equipment according to the planning information.

Herein, the position sensor is embodied to record sensor data describing the orientation of the biopsy needle in three dimensions. The position sensor can in particular be a gyroscopic sensor, but can also be embodied otherwise.

Therefore, it is proposed according to one or more example embodiments that a hand-held biopsy device comprising the biopsy needle and a position sensor be combined with marking equipment such that highly accurate positioning of the biopsy needle for removing the tissue sample is nevertheless possible in a space-saving and cost-effective manner on the basis of planning information based on image data from the imaging equipment. Here, outputting equipment is used that informs the person performing the biopsy in such a way that the biopsy needle can be aligned according to the target orientation. The marking equipment provides information about the target entry point.

This enables the biopsy equipment to be implemented in an extremely cost-effective and convenient way since no motors, power electronics or movable axis guides and/or precision guides are required. The biopsy device can be implemented with a very low weight and can therefore be easily guided by the person performing the biopsy, in particular for setting the target orientation. Since the hand-held biopsy device can be removed from the object-support table and in particular does not have to be attached to the object-support table or the stand, as is the case with a highly complex heavy biopsy unit in the prior art, there are hardly any space restrictions compared to imaging equipment alone and free access to the patient is fully maintained. At the same time, patients feel very safe, since the procedure is carried out manually by a human being.

Samples can be removed quickly, as is also the case with ultrasound-guided biopsies, for example. Since it is not necessary to install a biopsy unit, no changeover is required between the screening and the biopsy. Here, it should be noted that the proposal according to one or more example embodiments also allows existing imaging equipment, in particular mammography equipment, to be retrofitted in a simple manner to form biopsy equipment, since ultimately only the marking equipment and the hand-held biopsy device have to be added. Control equipment and a display or the like are generally already present.

In actual operation, whenever planning information is available that can, for example, describe a desired path of the biopsy needle, the entry point is transmitted to the marking equipment or the marking equipment is actuated according to the entry point to be marked. In other words, the control equipment as a whole can be embodied to transmit the entry point to the marking equipment and/or to actuate marking equipment according to the entry point.

In particular, the marking equipment can comprise a marking laser. It is therefore conceivable to mark the entry point with light on the surface of the patient. For this purpose, the use of laser light is preferred due to its good identifiability, wherein, however, other types of projection equipment can be used. An example of suitable marking equipment was, for example, described in the publication “Integration eines Projektors zur Einblendung von Positionier- und Untersuchungshilfen” [“Integration of a Projector for Superimposing Positioning and Examination Aids” ] in Prior Art Journal 2020 #05—Pages: 119-122—ISBN: 978-3-947591-33-6; Volume No.: 99.

Expediently, the marking equipment can be arranged on the imaging equipment. Arranging the marking equipment on the imaging equipment enables registration in a particularly simple manner. Specifically and particularly preferably, herein, the marking equipment can be arranged on or integrated in a radiator unit that includes the X-ray source of the imaging equipment. In mammography equipment and known biopsy equipment, it is known to use the X-ray detector with a cover itself as the object-support table, wherein the breast can be compressed and held in position via an additional upper compression plate (also referred to as a compression paddle). The X-ray source is arranged above the object-support table and the compression plate in the radiator unit, movably if tomosynthesis is possible, from where the target entry position can be marked in an excellent manner.

In this context, it is also particularly advantageous for the compression equipment to comprise the upper compression plate that compresses the breast and in particular faces away from the X-ray detector of the imaging equipment, wherein the compression plate comprises at least one feed-through opening for passing through the biopsy needle. As already mentioned, herein, the lower edge of the breast preferably rests on a detector cover of the X-ray detector which forms the object-support table. Therefore, an upper compression plate is used which allows access to the breast, at least in regions defined by the feed-through openings, in particular at least one window. These feed-through openings can, on the one hand, be used to project the entry point, preferably, as mentioned, from above and, on the other hand, to gain access with the biopsy needle of the biopsy device. In a specific embodiment, it can be provided that the compression plate comprises a plurality of feed-through openings arranged in a matrix. Such a compression plate can also be referred to as an “alphanumeric compression plate” since a type of “checkerboard pattern” can be provided for the feed-through openings and the individual feed-through openings can be identified with a letter (for one direction) and a number (for the other direction).

In a development of one or more example embodiments, it can be provided that it comprises a holder for the biopsy device, in particular arranged on the imaging equipment. For example, the holder can be arranged on the side of the imaging equipment, in particular on a stand of the imaging equipment, but other arrangement positions are also conceivable. The biopsy device can be stored in the holder when it is not needed, for example, when the imaging equipment is to be used for screening. Expediently, the holder can serve various other functions.

For example, it can be specifically provided that the holder is embodied to establish a predetermined calibration orientation of the biopsy device for calibrating the position sensor. This can, for example, be achieved via shaping and/or on the basis of suitably selected support surfaces. In particular, the holder can be embodied such that the biopsy device can only be inserted therein with a precise fit in the specific calibration orientation. Therefore, whenever the biopsy device is inserted into the holder, the position sensor can be recalibrated so that the position information can always be ascertained in a clearly defined manner in a way registered with the imaging equipment.

The holder can further comprise an electrical interface for charging an electrical energy store of the biopsy device. Here, the interface can, for example, include connecting means on the holder side that interact with connection means on the biopsy device side. In particular when the biopsy device is inserted into the holder, the electrical connection for charging the electrical energy store is also established directly. In this sense, the holder can also be understood as a type of “charging cradle”.

Generally speaking, it is also expedient for the biopsy device to be embodied as wireless, i.e., not to require any connection cables, that could interfere with and/or hinder the biopsy. Specifically, it can be provided that, in particular in addition to the energy store, the biopsy device comprises communication equipment for wireless communication with the control equipment. For example, the communication equipment can be Bluetooth equipment.

Preferably, the biopsy device can comprise a control unit that can communicate with the control equipment via the communication equipment, in particular bidirectionally. The control unit controls the operation of the biopsy device. It can in particular be embodied to ascertain the position information from the sensor data from the position sensor and
to transmit the position information or information derived therefrom to the control equipment (external to the biopsy device) via the communication equipment and/or
to ascertain a deviation of the orientation of the biopsy needle from the target orientation obtained via the communication equipment.

Here, it is in particular expedient both for the control equipment to be informed about the current position information and for the control unit to know the target orientation. This is because, as will be explained in more detail below, it is then conceivable for the outputting equipment to be ultimately implemented in a distributed manner in order to provide the person performing the procedure with support instructions as guidance information in a variety of ways.

In a particularly preferred embodiment of the present invention, it is provided that the biopsy device comprises output means of the outputting equipment, which is embodied to output a support instruction in dependence on the relative orientation of the biopsy needle according to the position information to the target orientation. If the output means is provided directly on the biopsy device, the attention of the person performing the biopsy does not have to be diverted from the biopsy device and the region of the procedure, in particular also the marked entry point, thus allowing full concentration on the procedure in a simplified manner. Herein, the output means can expediently be actuated by the above-described control unit if, as described above, the deviation from the target orientation is ascertained within the biopsy device. Therefore, in this case, only the target orientation needs to be provided to the biopsy device by the control equipment, for which purpose the communication equipment can be used, so that the biopsy device can autonomously guide the person performing the biopsy to adopt the target orientation via corresponding outputs via the output means, while the entry point is indicated externally by the marking equipment registered with the imaging equipment.

Herein, particularly preferably, the output means can be haptic output means. Specifically, the haptic output means can be a vibration motor. In this way, the person performing the procedure can continue to focus the gaze on the actual biopsy region, in particular the entry point and the biopsy needle, since the output means is directed at other senses. Since the biopsy device is hand-held, vibrations are very noticeable. Additionally or alternatively, the output means can also be acoustic output means. Optical output means is also conceivable, but less preferable, at least on its own. Herein, it is in principle conceivable to provide feedback whenever the biopsy needle is located in the target orientation. However, it is preferable to use the feedback via the output means, in particular the haptic output means, in order to guide the person performing the biopsy step-by-step to the target orientation.

Therefore, in a specific, advantageous embodiment, the output means can be actuated according to a target range, which decreases over time, to be set around the target orientation in order to guide the person performing the biopsy step-by-step to the target orientation. Therefore, in particular the control unit can be embodied to perform this actuation. In other words, it is proposed that a large “capture region” is changed to a smaller “capture region”. Here, it can be specifically provided that the output means is activated on leaving the target range. This means that whenever the person performing the biopsy looks likely to leave the target range, said person is guided back into said range by corresponding feedback. Thus, as the target range decreases over time, the target orientation is adopted more and more precisely, first by guidance to a rough target range, which is then increasingly narrowed. Thus, particularly intuitive guidance to the target orientation is provided.

There are other conceivable approaches for making specific use of the output means, for example, in the case of an acoustic output means, to guide the user in the manner of a “parking aid” to the target orientation by emitting temporally spaced apart tones at a greater distance from the target orientation, wherein the temporal space between them decreases until a passing tone is present when the target orientation is reached.

Alternatively, or preferably additionally, it can be provided that the outputting equipment includes a display, wherein the control equipment actuates the display to output a representation visualizing the relative orientation of the needle according to the position information to the target orientation. Therefore, the representation then forms the guidance information. Here, it is, for example, possible to use a display that is also used by the imaging equipment to represent image data and the like. The display can also be a touchscreen, which can also be used as input equipment. Preferably, the display is or can be arranged in a viewing area of the person performing the biopsy, for example via adjustable holding equipment, such as, for example, a holding arm.

Here, the display is preferably controlled directly by the control equipment, which receives the position information from the biopsy device via the communication equipment. The graphical representation can in particular contain an abstracted and/or schematic representation of the biopsy needle in the current orientation described by the position information compared to the target orientation represented by an additional element. This representation can, for example, be ascertained as a superimposition of image data, in particular image data on which the planning information is based. However, abstraction is also possible with respect to the background, in particular since, in contrast to its orientation, the current actual position of the biopsy needle is generally not known and thus confusion can be avoided.

The biopsy equipment can further comprise planning equipment for at least partially automatically ascertaining the planning information from image data from the imaging equipment. Herein, the imaging equipment can, for example, record two- or three-dimensional scout scans which can be at least partially automatically evaluated by the planning equipment, which can, for example, include a workstation. For example, display equipment of the workstation can show a planning user interface via which a user can intervene manually in the planning, for example to mark the region of interest and the like. Planning procedures and general procedures for ascertaining the planning information are already largely known in the prior art and will not be explained in detail here.

Preferably, the biopsy needle can comprise markings for displaying the depth of penetration into a patient, in particular along the needle shaft. For example, markings can be provided at regular intervals along the needle shaft of the biopsy needle so that the current penetration depth can be easily read by the person performing the biopsy. The target penetration depth according to the planning information can, for example, be output via the outputting equipment, in particular the display, for reference. In addition to the parameters ‘entry point’ and ‘target orientation’, such markings also enable the target penetration depth to be monitored and checked. Herein, it should be noted that, alternatively or additionally, if corresponding detection means are provided in the biopsy device, a current penetration depth can also be output on an output means or the output means of the biopsy device. It is also in principle conceivable for an actuator to be provided in the biopsy device for automatically extending the biopsy needle, but this is less preferable, since, as mentioned, actuators are preferably avoided in order to keep the biopsy device manageable and light.

In addition to the biopsy equipment, the present invention also relates to a method for supporting a person performing a biopsy with biopsy equipment according to one or more example embodiments, wherein the outputting equipment is actuated to output guidance information indicating the relative orientation of the needle according to the position information to a target orientation according to the planning information. All statements relating to the biopsy equipment according to one or more example embodiments, in particular with regard to communication, control by the control equipment and the control unit and the specific embodiment of the outputting equipment, can be applied analogously to the method according to one or more example embodiments with which, therefore, the advantages already mentioned can also be obtained.

Finally, one or more example embodiments also relates to a hand-held biopsy device for biopsy equipment according to one or more example embodiments, comprising a biopsy needle and a position sensor for measuring position information indicating the orientation of the biopsy needle. Where applicable, the statements relating to the biopsy equipment and the method also apply analogously to the biopsy device. In particular, the biopsy device can therefore also comprise the control unit, the electrical energy store, the communication equipment and/or connection means for charging the electrical energy store. However, particularly preferably the biopsy device comprises output means, which, as described above, can in particular be actuated by the control unit in order to display the relative orientation to or deviation from a target orientation, in particular obtained via the communication equipment, for example according to a target range around the target orientation which becomes smaller over time.

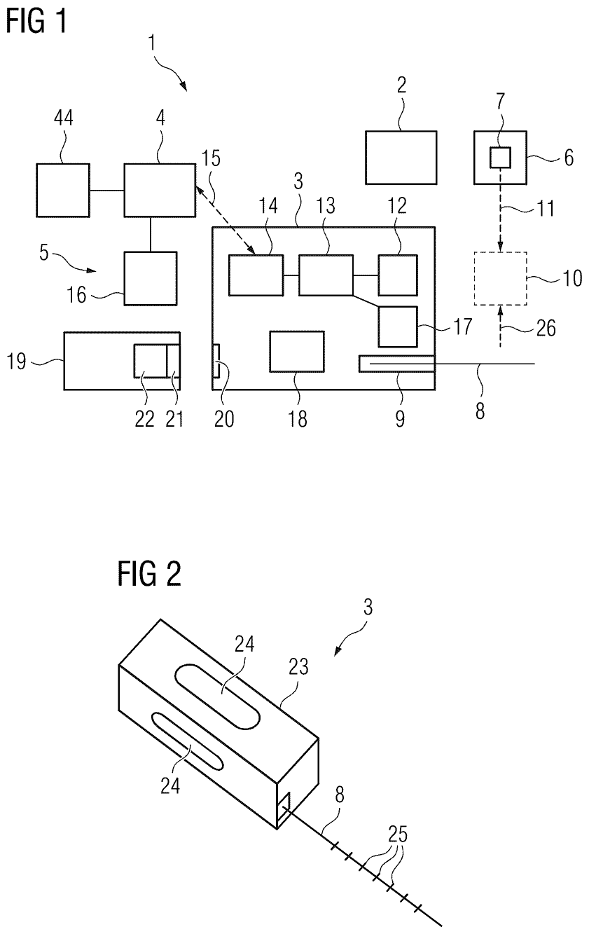

FIG. 1 shows the functional structure of biopsy equipment 1 according to one or more example embodiments. The biopsy equipment 1 includes as main components imaging equipment 2, which is not itemized further here, a hand-held wireless biopsy device 3, control equipment 4, outputting equipment 5, which in the present case is divided into a plurality of components, and marking equipment 6, which in the present case is attached to the imaging equipment 2 or integrated therein. In addition, in the present case, the biopsy equipment 1 also includes planning equipment 44, which can, for example, be embodied as a workstation.

The planning equipment 44 is embodied to perform planning, at least partially automatically, based on image data from the imaging equipment 2, in the present case X-ray equipment for tomosynthesis of the female breast, in which a user can also be involved. The result of the planning is planning information, which in particular indicates how a biopsy needle 8 of the biopsy device 3, which is attached there via a needle holder 9, is to access a region of interest where a tissue sample is to be taken within the breast 10 currently to be treated, which is indicated schematically here. Therefore, the planning information in particular describes a target entry point, a target orientation and also a target penetration depth of the biopsy needle 8. This planning information is provided to the control equipment 4.

In the present case, the control equipment 4 forwards the entry point to the marking equipment 6 which comprises a marking laser 7 and is registered with the imaging equipment 2 with a coordinate system to which the planning information also refers. Therefore, the marking equipment 6 can project the entry point with high precision onto the breast 10 for a person performing the biopsy according to the dashed arrow 11.

In order also to be able to set the target orientation correctly, the biopsy device 3 comprises a position sensor 12, for example a gyroscopic sensor, which can measure the current orientation of the biopsy device 3 in all three spatial directions. The sensor data from the position sensor 12 is evaluated by a control unit 13 of the biopsy device 3 to produce position information. The biopsy device 3 can communicate wirelessly with the control equipment 4 via communication equipment 14, as indicated by the arrow 15. In the present case, the communication equipment 14 is embodied as a Bluetooth module and the control equipment 4 is embodied as Bluetooth-capable. The control unit 13 receives the target orientation provided from the control equipment 4 via the communication equipment 14 so that it can identify a deviation of the current orientation of the biopsy device 3, and thus the biopsy needle 8, from the target orientation. At the same time, the control unit 13 can provide the position information to the control equipment 4 via the communication equipment 14.

The control equipment 4 and the control unit 13 use different components of the outputting equipment 5 to give the person performing the biopsy, who is holding the biopsy device 3 in the hand, support instructions as guidance information that guides said person to adopt the target orientation of the biopsy needle 8.

Specifically, initially the biopsy equipment 1 generally comprises a display 16 as part of the outputting equipment 5 on which a graphical representation visually displays a potential deviation from the target orientation. Such a representation can be easily generated by the control equipment 4 based on the position information.

The biopsy device 3 further comprises output means 17 as a further component of the outputting equipment 5, in the present case haptic output means embodied as a vibration motor. For example, support instructions can be output directly without said person having to divert the gaze from the site of the intervention, as will be explained in more detail below. The support instructions are output in dependence on the deviation from the target orientation ascertained by the control unit 13.

In the present case, the control unit 13 is embodied as a microcontroller and comprises firmware for executing the functions described.

In order to be able to supply the components of the biopsy device 3 with electrical energy, the biopsy device 3 further comprises a rechargeable electrical energy store 18, for example a battery. As a result of the wireless communication via the communication equipment 14 and the integrated electrical energy supply, the biopsy device 3 is wireless.

When it is not needed, the biopsy device 3 can be stored in a holder 19, which can, for example, be arranged on a stand of the imaging equipment 2. The holder is embodied such that the biopsy device 3 can only be inserted into the holder 19 in a predetermined calibration orientation. Therefore, whenever the biopsy device 3 is inserted in the holder 19, the position sensor 12 can be recalibrated since the calibration orientation is then known. It is possible to identify that this is the case, for example, by a connection of connection means 20 of the biopsy device 3 to connection means 21 of an electrical interface 22 for charging the energy store 18 also being established on the part of the holder 19, which preferably takes place automatically when the biopsy device 3 is inserted into the holder 19.

FIG. 2 is a perspective view of a possible embodiment of the biopsy device 3. The components of the biopsy device 3 shown in FIG. 1 are accommodated in a housing 23, which can comprise handling means 24, for example handles, for improved handling of the biopsy device 3. As can also be seen in FIG. 2, markings 25 are provided at regular intervals along the needle shaft of the biopsy needle 8 at which the depth of penetration of the biopsy needle 8 into the breast 10 can be read, see also arrow 26 (FIG. 1).

Since the target penetration depth is also displayed on the display 16, this can also be easily adhered to by the person performing the biopsy. Further output means can also be provided on the biopsy device 3 in order to display the target penetration depth.

FIG. 3 shows a view of the imaging equipment 2, in the present case X-ray equipment embodied for tomosynthesis of the breast 10. It therefore includes a stand 27 on which a radiator unit 28 with the X-ray source 29 and an X-ray detector 30 with a cover 31 as an object-support table 32 are arranged. Together with the detector 30 and its cover 31, an upper displaceable compression plate 33 forms compression equipment 43 for the breast 10.

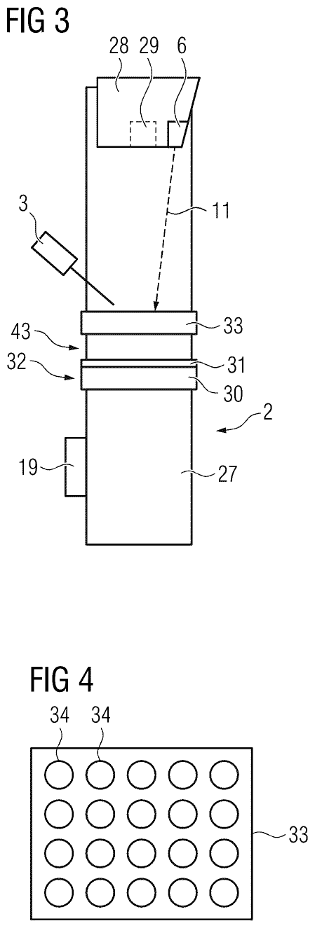

To enable the biopsy to take place through the compression plate 33 (often also called a compression paddle), it comprises, as the top view in FIG. 4 shows, feed-through openings 34, which in the present case are arranged in a matrix. The planning information can indicate which feed-through openings 34 are to be used. The feed-through openings 34 also allow the entry point to be projected onto the surface of the breast 10 according to the dashed arrow 11. They further allow the biopsy needle 8 to be inserted into the breast 10 (dashed arrow 26).

For a specific biopsy according to planning information, it can now be provided, for example, that the control equipment 4 transmits the entry point to the marking equipment 6 and actuates this to project the entry point onto the surface of the breast 10. The person performing the biopsy can now take the biopsy device 3 in the hand and place the biopsy needle 8 on the entry point or insert it slightly. The person then uses the support instructions of the outputting equipment 5 to adopt the target orientation and subsequently the markings 25 of the biopsy needle 8 to select the correct penetration depth corresponding to the target penetration depth.

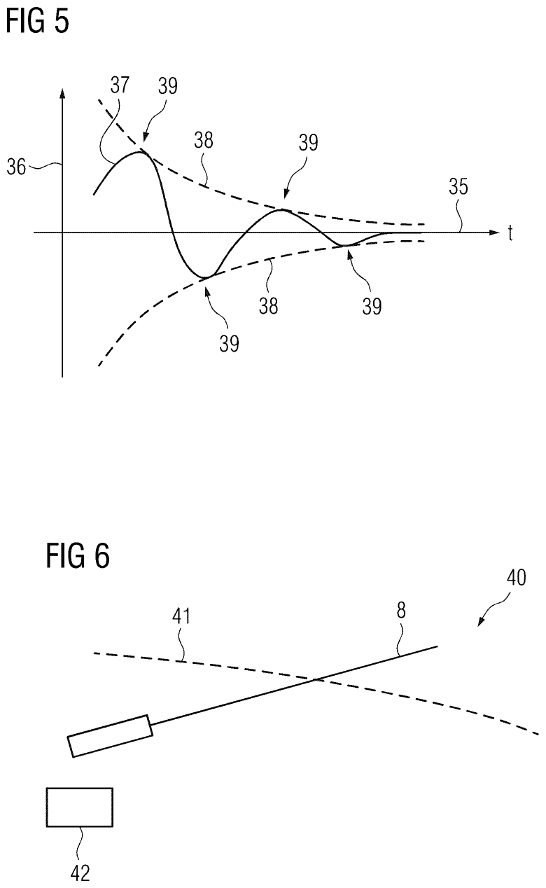

Specific variants for supporting the person performing the biopsy in adopting the target orientation are explained with reference to FIGS. 5 and 6. The two variants are preferably used in a complementary manner.

The first variant uses the haptic output means 17 of the biopsy device 3. In this respect, FIG. 5 shows the time profile along the abscissa 35 and the deviation of the current orientation of the biopsy needle 8 from the target orientation along the ordinate 36. The solid graph 37 shows the time profile of the setting made by the person performing the biopsy, while the dashed graphs 38 show the profile of a target range around the target orientation that narrows over time (i.e. deviation zero). Whenever the deviation reaches the edge of the target range, see points 39, haptic feedback is output via the output means 17, so that, as can be seen from the profile 37, the user is intuitively and naturally guided over time, from a coarse adjustment to a fine adjustment, to the target orientation.

FIG. 6 shows a possible visual graphical representation 40 on the display 16. The position information can be used to display the position of the biopsy needle 8, shown here in stylized form, to the target orientation 41, shown by a dashed line. The visual representation 40 can be updated in real time and can also be supplemented by further elements, in particular a display 42 of the target penetration depth, which is also only indicated schematically here. The representation 40 can be in color.

Although the invention has been illustrated and described in detail by the preferred exemplary embodiment, the invention is not restricted by the disclosed examples and other variations can be derived herefrom by the person skilled in the art without departing from the scope of protection of the invention.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Independent of the grammatical term usage, individuals with male, female or other gender identities are included within the term.

1. Biopsy equipment for performing a biopsy on a female breast, comprising:
imaging equipment for tomosynthesis of the breast, the imaging equipment including compression equipment configured to fix the breast;
a hand-held biopsy device including a biopsy needle, the biopsy device including a position sensor configured to measure position information indicating an orientation of the biopsy needle;
marking equipment registered with the imaging equipment, the marking equipment configured to mark an entry point for the biopsy needle on the breast fixed in the compression equipment according to planning information for a biopsy to be performed based on image data from the imaging equipment;
outputting equipment configured to output guidance information indicating a relative orientation of the biopsy needle according to the position information to a target orientation according to the planning information for a person performing the biopsy; and
control equipment configured to control the biopsy equipment according to the planning information.

2. The biopsy equipment of claim 1, wherein the marking equipment at least one of comprises a marking laser or is arranged on the imaging equipment.

3. The biopsy equipment of claim 1, wherein the compression equipment comprises an upper compression plate, the upper compression plate is configured to compress the breast and faces away from an X-ray detector of the imaging equipment, and the upper compression plate includes at least one feed-through opening for passing through the biopsy needle.

4. The biopsy equipment of claim 1, wherein further comprising:
a holder for the biopsy device.

5. The biopsy equipment of claim 4, wherein the holder at least one of is configured to establish a predetermined calibration orientation of the biopsy device to calibrate the position sensor or comprises an electrical interface configured to charge an electrical energy store of the biopsy device.

6. The biopsy equipment of claim 1, wherein the biopsy device comprises communication equipment configured to wirelessly communicate with the control equipment.

7. The biopsy equipment of claim 1, wherein the biopsy device comprises output means of the outputting equipment, the output means configured to output a support instruction in dependence on the relative orientation of the biopsy needle according to the position information to the target orientation.

8. The biopsy equipment of claim 7, wherein the output means is a haptic output means.

9. The biopsy equipment of claim 7, wherein the output means is configured to be actuated according to a target range that decreases over time, the target range is around the target orientation to guide the person performing the biopsy step-by-step to the target orientation.

10. The biopsy equipment of claim 9, wherein the output means is activated on leaving the target range.

11. The biopsy equipment of claim 1, wherein the outputting equipment includes a display, wherein the control equipment is configured to actuate the display to output a representation visualizing the relative orientation of the biopsy needle according to the position information to the target orientation.

12. The biopsy equipment of claim 1, further comprising:
planning equipment configured to at least partially automatically ascertain the planning information from the image data from the imaging equipment.

13. The biopsy equipment of claim 1, wherein the biopsy needle comprises markings displaying a depth of penetration into a patient.

14. A method for supporting a person performing a biopsy with the biopsy equipment of claim 1, comprising:
actuating the outputting equipment to output guidance information indicating the relative orientation of the biopsy needle according to the position information to the target orientation according to the planning information.

15. A hand-held biopsy device for the biopsy equipment of claim 1, comprising the biopsy needle and the position sensor configured to measure the position information indicating the orientation of the biopsy needle.

16. The biopsy equipment of claim 4, wherein the holder is on the imaging equipment.

17. The biopsy equipment of claim 13, wherein the markings are along a shaft of the biopsy needle.

18. The biopsy equipment of claim 2, wherein the compression equipment comprises an upper compression plate, the upper compression plate is configured to compress the breast and faces away from an X-ray detector of the imaging equipment, and the upper compression plate includes at least one feed-through opening for passing through the biopsy needle.

19. The biopsy equipment of claim 7, wherein the output means is configured to be actuated according to a target range that decreases over time, the target range is around the target to guide the person performing the biopsy step-by-step to the target orientation.

20. The biopsy equipment of claim 19, wherein the output means is activated on leaving the target range.
